(19) United States
(12) Patent Application Publication
(10) Pub. No.: US 2022/0249870 A1
(43) Pub. Date: Aug. 11, 2022

(54) MULTI-LEAF COLLIMATOR FOR RADIOTHERAPY APPARATUS AND RADIOTHERAPY APPARATUS USING THE SAME

(71) Applicants: Weijie CUI, Beijing (CN); Accelink Technologies Co., Ltd., Beijing (CN)

(72) Inventors: Jianrong DAI, Beijing (CN); Weijie CUI, Beijing (CN)

(21) Appl. No.: 17/667,518

(22) Filed: Feb. 8, 2022

(30) Foreign Application Priority Data
Feb. 10, 2021 (CN) CN 202110184691.5

(51) Int. Cl. A61N 5/10; G21K 1/04

(52) U.S. Cl. CPC A61N 5/1045; G21K 1/046

(57) ABSTRACT

A multi-leaf collimator comprises a plurality of pairs of leaves, leaves of each pair being disposed oppositely from each other. Each pair of leaves comprises a first leaf and a second leaf which are configured to move oppositely relative to each other. In a first plane perpendicular to an isocenter plane of the radiotherapy apparatus and parallel to a direction of leaf movement, the first leaf and the second leaf are capable of moving to a closed position, where end surfaces of the first leaf and the second leaf are embedded with each other at the closed position.

### CROSS-REFERENCE TO RELATED APPLICATIONS

The present application claims the benefit of, and priority to, a Chinese patent application No. 202110184691.5, entitled “MULTI-LEAF COLLIMATOR FOR RADIOTHERAPY APPARATUS AND RADIOTHERAPY APPARATUS USING THE SAME”, filed on Feb. 10, 2021, the disclosure of which is hereby expressly incorporated by reference herein in its entirety.

### TECHNICAL FIELD OF THE DISCLOSURE

The present disclosure relates to a field of radiotherapy equipment, and in particular, to a multi-leaf collimator, and a radiotherapy apparatus with a corresponding collimator.

### BACKGROUND

The multi-leaf collimator (MLC) is an important component of contemporary radiotherapy machines. It is installed directly under the head of the radiotherapy machine and is used for limiting the irradiation range of the radiation beam, thus forming an irregular radiation field suitable for various target area shapes, so as to realize conformal radiotherapy. In the irradiation process, the position of the leaves of a multi-leaf collimator can be adjusted, so that the radiation intensity distribution of the irradiation range may be adjusted to perform an intensity modulated radiotherapy. Compared with conventional radiotherapy, conformal radiotherapy and intensity-modulated radiotherapy can better achieve the purpose of radiotherapy, i.e. killing tumors as much as possible while protecting the surrounding normal tissues.

The collimator leaf is a basic unit of a multi-leaf collimator, which is made of a heavy metal material (e.g., tungsten alloy) with a long strip shape. The length of a leaf is determined by the largest radiation field which needs to be formed; the width is ranged from several millimeters to several centimeters, and the narrower the width, the more suitable the formed radiation field may be for the target area; the thickness is at least five half value layers of the used metal material, so that the radiation of the leaf shielding area is attenuated below 5% of original intensity. Each leaf is driven by an independent motor, and a plurality of leaves is arranged adjacently and tightly to form a leaf set.

For the design of the multi-leaf collimator and the leaves thereof, researchers have provided a plurality of different improved solutions from different aspects, and some solutions have been applied in manufacturing and use. One option is to design the arrangement mode of the leaves, ranging from conventional single-layer to double-layer or even three-layer arrangement. Another improved design is to optimize the width and the shape of leaf end projected on the isocenter plane, through modifying the conventional equal-width leaf and the straight end surface, and obtaining the optimum leaf width and end shape through the optimization method.

There are two issues that needs to be taken into special consideration with respect to the cross-sectional shape of the leaf end in a plane perpendicular to the isocenter plane and parallel to the movement direction of the leaf. When a pair of leaves are in an open state, the leaf end is in a transition area between the area where the radiation is blocked from the leaves and the open irradiation area, so the end design has an influence on the dosage of the transition area, i.e. the penumbra. A focusing design is preferably used to make the penumbra as small as possible and minimize the change thereof along with different positions of the leaf. In a case where the cross-sectional shape of the leaf end is a linear segment, in order to achieve a focusing effect, the leaf must move along a circular arc trajectory centered on the radiation source; if the leaf moves along a linear trajectory in a direction vertical to the central axis of the radiation beam, the leaf needs to rotate itself by a small angle after it reaches the designated position, to make the straight end surface tangent to the radiation beam.

Another issue related to the leaf end design is that there is always a slit when opposite leaves are closed, leading to the problem of collision and leakage radiation. If the slit is too small, the leaves are likely to collide; if the slit is too large, it will leak too much radiation and cause side effects during irradiation. For example, the minimum projection width of the leaf slit on the isocenter plane of the Varian system is 0.05 cm, while the minimum projection width of the leaf slit on the isocenter plane of the Elekta system is 0.5 cm. For the non-focused end surface, a wider gradual change area of dose is formed below the leaf end when the opposite leaves are closed. The leakage radiation caused by the leaf slit and the gradual change area of dose below the leaf end not only causes normal tissue to be subjected to unnecessary irradiation, but also increase the difficulty for modeling and dose calculation of the accelerator, which may increase the dose calculation error.

### SUMMARY

In order to overcome the defects in the prior art, the present disclosure proposes improving the multi-leaf collimator through modifying the end design of the leaf of the multi-leaf collimator. In particular, regarding the problem that the opposite leaves of an existing collimator cannot be completely closed and a slit exists between the leaves, the present disclosure proposes an apparatus to allow the opposite leaves be embedded with each other through designing the end of leaves of the multi-leaf collimator, thus improving the working performance of the multi-leaf collimator.

According to an exemplary embodiment, there is provided a multi-leaf collimator for a radiotherapy apparatus, comprising a plurality of pairs of leaves, leaves of each pair being disposed oppositely from each other. Each pair of leaves comprises a first leaf and a second leaf which are configured to move oppositely relative to each other. In a first plane perpendicular to an isocenter plane of the radiotherapy apparatus and parallel to a direction of leaf movement, the first leaf and the second leaf are capable of moving to a closed position, where end surfaces of the first leaf and the second leaf are embedded with each other at the closed position.

In one embodiment, the end surface of the first or second leaf is configured as a fold line contour composed of a plurality of recesses and a plurality of protrusions.

In one embodiment, the plurality of recesses and the plurality of protrusions of the end surface are alternately distributed.

In one embodiment, contour lines of the plurality of protrusions of the end surface of the first or second leaf are formed as an arc segment.

In one embodiment, projections of the plurality of recesses and the plurality of protrusions of the end surface in a second plane perpendicular to the isocenter plane and the direction of leaf movement are disposed longitudinally and alternately along a direction of a connection line connecting a radiation source and a center of the multi-leaf collimator.

In one embodiment, the projections of the plurality of recesses and the plurality of protrusions of the end surface in a second plane perpendicular to the isocenter plane and the direction of leaf movement are disposed in a lattice-like arrangement.

In one embodiment, an end portion of the first leaf and/or the second leaf has a first thickness in a direction of a connection line connecting a radiation source and a center of the multi-leaf collimator, a main portion other than end portion of the first leaf and/or the second leaf has a second thickness, and the first thickness is greater than the second thickness.

In one embodiment, the first thickness of the first leaf and/or the second leaf decreases to the second thickness along a length direction from the end surface to the main portion of the first leaf and/or the second leaf.

In one embodiment, an embedment depth between the end surface of the first leaf and the second leaf at the closed position can be calculated according to the distance from an embedment position to the center line of the pair of leaves. Preferably, in a case where the end surface of the first leaf and end surface of the second leaf are arc-shaped, the embedment depth between the end surfaces of the first leaf and the second leaf at the center line of the pair of leaves is twice larger than an a contour height of an arc segment of the end surface of the first leaf or the second leaf. The embedment depth may decrease towards both the top and bottom edges of the leaf, and the embedment depth at the edges is greater than zero.

According to an exemplary embodiment, provided is a radiotherapy apparatus, comprising: a multi-leaf collimator comprising a plurality of pairs of leaves, leaves of each pair being disposed oppositely from each other and comprising a first leaf and a second leaf configured to move oppositely relative to each other, wherein, in a first plane perpendicular to an isocenter plane of the radiotherapy apparatus and parallel to a direction of leaf movement, the first leaf and the second leaf are capable of moving to a closed position, and end surfaces of the first leaf and the second leaf are embedded with each other at the closed position; and a controller, which is configured to control an embedment degree of the end surfaces of the first leaf and the second leaf at the closed position, so that the first leaf and the second leaf do not collide at the closed position.

The beneficial effect of the present disclosure is that at the closed position, the opposite first leaf and the second leaf are embedded with each other in the direction of leaf movement, so that the slit between the ends of the pair of leaves occurring when the leaves are closed for the conventional collimator no longer exists. Therefore, the shielding capability against the radiation beams can be enhanced, and the leakage radiation and the dose gradual change area are reduced. Meanwhile, the degree of embedment between the leaves can be adjusted so that there will be no collision when the leaves are closed. Compared to a conventional collimator, the range of leaf adjustment is increased, and operation is easier. The problems of the collision, the leakage radiation and the dose gradual change area of the opposite leaves during the use of the multi-leaf collimator has been solved in the present disclosure, so the collimator improves conformal intensity adjustment effect, and the difficulty of modeling and planning optimization of the collimator is also reduced.

### BRIEF DESCRIPTION OF THE DRAWINGS

Through a more detailed description of the embodiments of the present disclosure in conjunction with the accompanying drawings, the above and other objectives, features and advantages of the embodiments of the present disclosure will become more apparent. The drawings are included to provide a further understanding of the embodiments of the present disclosure, and are incorporated in and constitute a part of the specification to explain the present disclosure, but are not intended to serve as a definition of the limits of the present disclosure. In the drawings, the same reference numeral usually represents an identical component. It should be understood that the dimensions and sizes of the components shown in the drawings are not necessarily drawn to scale, and they may be different from those used in the embodiments for implementation shown herein. Furthermore, some embodiments may combine any suitable combination of features from two or more drawings.

FIG. 1 shows a schematic diagram of the structure and function of a multi-leaf collimator.

FIG. 2 shows a schematic diagram of a pair of opposite leaves of conventional design in different working states, which specifically shows a perspective view of the pair of leaves in a case of opening (a) and closing (b), a side view in a case of opening (c) and closing (d), and a front view (e) of end surfaces of the two leaves.

FIG. 3 shows a schematic diagram of a pair of leaves with an embedded end design in different working states according to an embodiment of the present disclosure, which specifically shows a perspective view of the pair of leaves in a case of opening (a) and closing (b), a side view of the leaves in a case of opening (c) and closing (d), and a front view (e) of end surfaces of the two leaves.

FIG. 4 shows the embedment depth (a) and the dimensions (b) and (c) of the end arc surface in a case where the opposite leaves are closed according to an embodiment of the present disclosure.

FIG. 5 illustrates a schematic diagram of a pair of leaves with an embedded end design in different working states according to another embodiment of the present disclosure, which specifically shows a perspective view of the pair of leaves in a case of opening (a) and closing (b), a side view of the leaves in a case of opening (c) and closing (d), and a front view (e) of end surfaces of the two leaves.

### DETAILED DESCRIPTION

The exemplary embodiments according to the present disclosure will be described hereinafter in detail with reference to the accompanying drawings. Obviously, the described embodiments are only a part of the embodiments of the present disclosure, rather than all the embodiments of the present disclosure, and it should be understood that the present disclosure is not limited by the exemplary embodiments described herein.

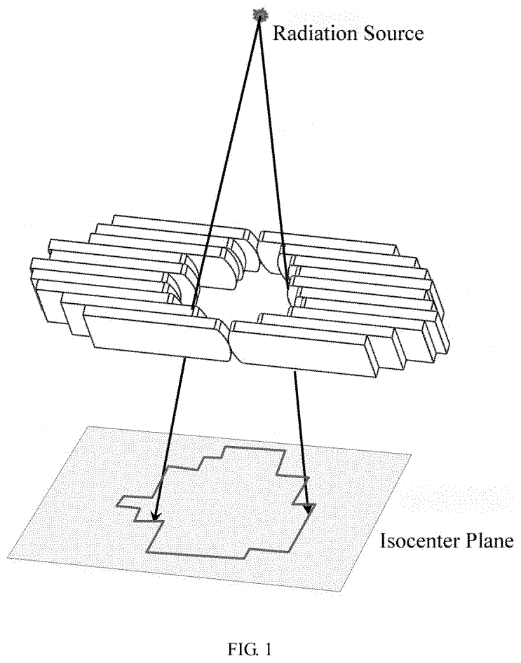

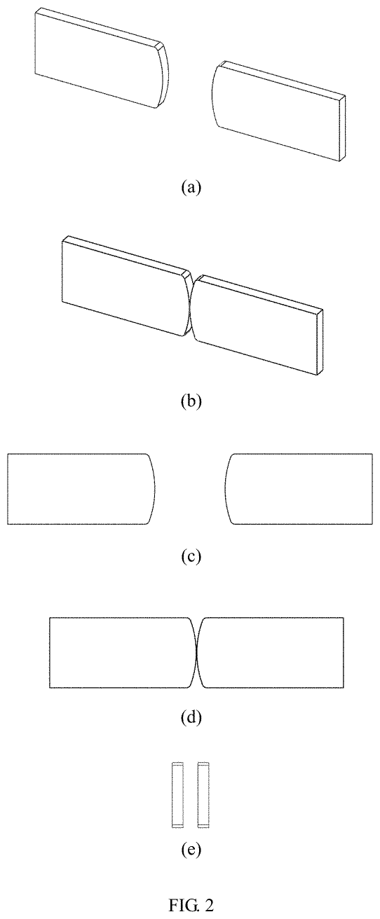

FIG. 1 is a schematic diagram of the structure and function of a multi-leaf collimator, which is installed in the treatment machine head of a radiotherapy apparatus. As shown in FIG. 1, the multi-leaf collimator is disposed below the radiation source and the collimator comprises two sets/arrays of leaves on the side A and the side B, which are symmetrically arranged above the isocenter plane of the treatment apparatus. Under the irradiation of the radiation source, the radiation beam passes through the slit between the leaf sets and reaches the tumor target area that needs to be irradiated. The leaves of the collimator can be driven to move independently through a driving mechanism such as a motor, so that different radiation fields can be formed in the isocenter plane. The contour of the radiation field determines the radiation area. If the radiation area is inconsistent with the expected irradiation range, some parts of the target area will not receive radiation, or the radiation beam may be irradiated to the nearby normal tissue.

FIG. 2 shows a schematic diagram of a pair of opposite leaves of conventional collimator in different working states, wherein (a) and (c) show the opposite position of the opposite leaves in an opening state, and (b) and (d) show the opposite position of the opposite leaves in a closing state. In the illustrated example, the projection of the leaf end surface in a plane perpendicular to the isocenter plane and parallel to the direction of the leaf movement has an arc shape. As described above, there is always a slit when the opposite leaves of the conventional collimator are closed. If the slit is too small, it is easy to collide, and if the slit is too large, the radiation leakage may occur, so it is difficult to overcome the problem of collision and radiation leakage at the same time.

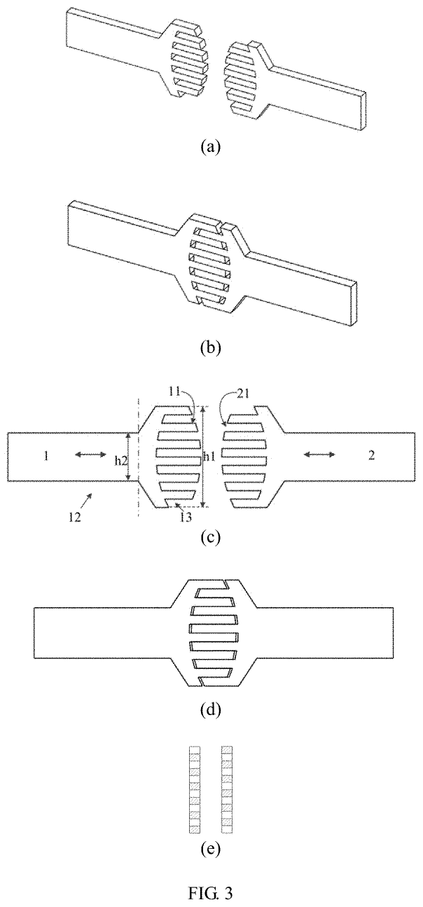

FIG. 3 shows a collimator with an embedded end design according to an embodiment of the present disclosure. In combination with FIG. 1 and FIG. 3, the multi-leaf collimator of the present disclosure comprises a plurality of pairs of leaves, and each pair of leaves are disposed oppositely from each other (only one pair of leaves is shown in FIG. 3). Each pair of leaves comprises a first leaf 1 and a second leaf 2, which can be controlled to move closely or apart from each other so as to block radiation or pass through the radiation. As shown in FIGS. 3 (a) and (c), the shapes of the first leaf 1 and the second leaf 2 present a point-symmetrical structure configuration. In addition, as shown in FIGS. 3 (b) and (d), in a plane perpendicular to the isocenter plane of the radiotherapy apparatus and parallel to the direction of leaf movement, i.e. the length direction of the leaf (hereinafter referred to as “first plane” for short), the first leaf 1 and the second leaf 2 can be moved and adjusted to a closed position. As shown in FIG. 3, the end surface 11 of the first leaf 1 and the end surface 21 of the second leaf 2 are embedded with each other at the closed position. Through such embedment design, the opposite leaves can effectively shield the radiation beams in a wider adjustment range under the closed state, which improves the safety and operability of the radiotherapy system.

In one embodiment, as shown in FIG. 3, the collimator leaf may be composed of a leaf main body 12 and a leaf end portion 13, and the end surface of the end portion 13 has a fold line contour composed of a plurality of recesses and a plurality of protrusions. According to one embodiment, on a cross section perpendicular to the isocenter plane and parallel to the leaf movement direction, the end surface contour of the end portion 13 is different from a linear segment or an arc segment of the conventional leaf, but a fold line segment composed of recesses and protrusions, where the recesses and the protrusions are alternately distributed. When the opposite leaves are closed, the recesses and protrusions of the two leaves are embedded with each other. For example, the protrusion of the leaf 1 is embedded in the corresponding recess of the leaf 2, and the protrusion of the leaf 2 is embedded in the corresponding recess of the leaf 1. Such a design divides the slit between the two opposite leaves into several small segments, and make the slit distributed in different positions along the radiation direction (i.e. the direction of the short-axis of the leaf). Thus, there is no longer leakage radiation directly passing through the slit, which can better solve the problem of radiation leakage and dose gradual change area occurring when the opposite leaves are closed.

In the example embodiment shown in FIG. 3, as shown in FIG. 3 (e), the projections of a plurality of recesses and a plurality of protrusions at the leaf end in a plane perpendicular to the isocenter plane and the direction of leaf movement (hereinafter referred to as the “second plane”) are arranged longitudinally and alternately along the direction of a connection line connecting the radiation source and the center of the collimator (e.g. the direction of the short-axis of the leaf). In the diagram, the blank area represents the protrusion, the shadow area represents the recess, and the protrusion and the recess are arranged in a longitudinal arrangement mode. The length of the protrusion and the recess in the longitudinal projection can be designed to be the same or different, which is not specifically limited in the present disclosure. Preferably, as shown in FIG. 3 (e), the length of projections of each protrusion and recess in the longitudinal direction is the same, so that the processability of the leaves can be improved.

The number of the protrusions and the recesses at the end of each leaf can be more than 2, for example, more than 4. Preferably, the number of protrusions at the end of each leaf is the same as the number of recesses. In the end design shown in FIG. 3, there are 6 protrusions and 6 recesses at the end of each leaf, while in actual manufacturing, the number of protrusions and recesses can be increased according to the processing capacity, so that the focusing ability of the leaves can be better retained.

It can be seen from FIG. 3 that the end of the leaf 1 and the leaf 2 uses the design with alternate distributions of recesses and protrusions. Meanwhile, the contour of a plurality of protrusions of leaf 1 or leaf 2 can also be formed as a specific shape, for example, a linear segment or an arc segment. Preferably, as shown in FIG. 3, the contour of a plurality of protrusions together forms an arc segment. In other words, the end surface contour at the protrusions is an interval arc formed by being truncated discontinuously on the end surface contour of the leaf shown in FIG. 2, which can improve the focusing performance of the leaf. Correspondingly, the contour of a plurality of recesses on the opposite leaf are also formed as an arc segments with the same contour, so that the two leaves can be embedded with each other in the closed position without collision. The radius of curvature of the arc segment may be determined according to the size of the leaf. For example, it can be between 8 and 30 cm. In a case where the protrusion has an arc contour, the extension length of each protrusion at the leaf end portion is also different. As shown in FIG. 3, the length of the protrusion gradually increases from the top edge of the leaf end portion along the radiation direction, and the protrusion at the central portion has a maximum length.

When the opposite leaves are closed, the protrusions and the recesses of the leaf 1 and the leaf 2 can be respectively embedded with each other. However, in a case where the leaves are opened, the recesses at the leaf end make the thickness of the leaf that attenuation radiation thinner, resulting in an increase of the amount of radiation penetration. Therefore, in one embodiment, the thickness h1 of the leaf end portion 13 in the direction of the connection line connecting the radiation source and the center of the collimator (i.e. the short axis direction of the leaf) may be set to be greater than the thickness h2 of the leaf main body 12. For example, h1 may be approximately twice the h2 to ensure that the thickness of the leaf that attenuate radiation at the leaf end is substantially the same as the thickness of the leaf main body (e.g. about 6-10 cm) if the end portion 13 and the main body 12 are prepared with the same material (e.g. tungsten alloy). Also, as shown in FIG. 3, the thickness of the leaf end portion 13 is not a constant value, which decreases to the thickness h2 along the direction from the leaf end surface 13 to the leaf main body 12. In other words, it increases from h2 along the direction of the leaf movement to h1 at the protrusion of the leaf. Viewed from the side, the collimator leaf designed in this way has a shape that is similar to the shape of a person's arm with open fingers.

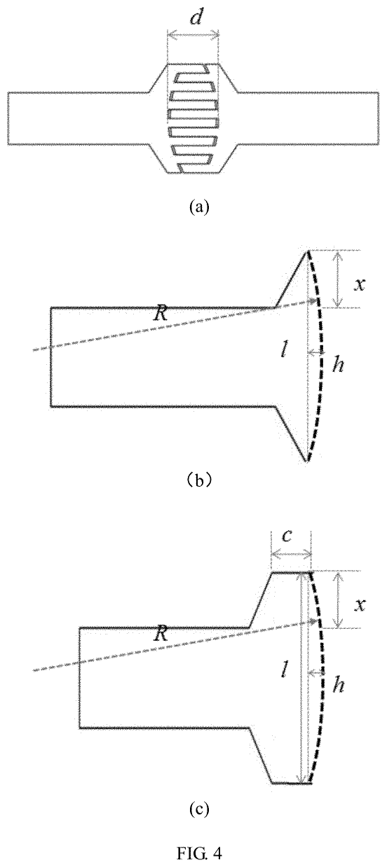

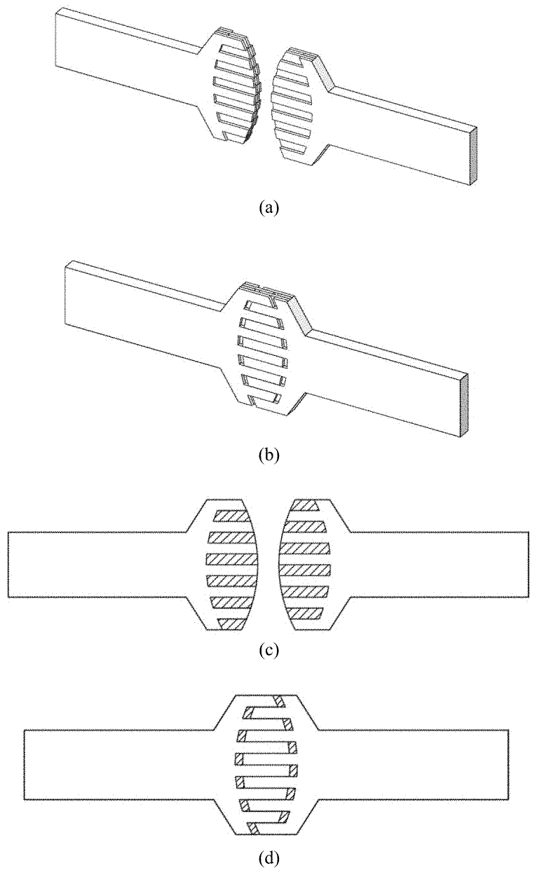

The depth of the recess of the end portion in FIG. 3 can be determined according to the size of the end portion and the embedment depth when the opposite leaves are closed. FIG. 4 shows a schematic diagram of the embedment depth when the opposite leaves are closed according to an embodiment of the present disclosure. As shown in FIG. 4 (a), the embedment depth between the two opposing leaves when they are closed can be defined as the distance between the vertices of the arc end surfaces of the two leaves, which can be calculated according to the distance from an embedment position to the lateral center line of the leaves. For the arc end surface shown in FIG. 4 (a), the embedment depth has a maximum value d at the lateral center line of the leaf, and it decreases toward the two edge sides. It can be understood that for other shapes of the leaf end, the embedment depth may have other values or distributions.

FIG. 4 (b) shows the design of leaf end and the corresponding size of the arc end surface according to one embodiment of the present disclosure. Assuming that the radius of the circular arc is R, the chord length corresponding to the circular arc is 1, and the contour height of the arc is h, the smaller of the distance between the position of the recess and the top surface and undersurface of the leave is x, then the depth y of the recess may be expressed as

y=d−2(R−\sqrt{R²−(l/2−x)²})

In a case where y derived according to the above formula is less than 0, it indicates that the embedment depth of the opposite leaves is relatively small, and there is no overlap between the end surfaces of the leaves when the leaves are closed, so the recess does not need to be set. In a case where the two circular arc end surfaces of the opposite leaves are exactly completely embedded, the embedment depth d should be equal to 2h, and it can be calculated that the recess depth at the center of the leaf (i.e. the extension length of the protrusion at the center of the leaf) is 2h, and the recess depth at the top/bottom edge of the leaf is 0. In consideration of keeping the radiation attenuation effect of the leaf as uniform as possible when the opposite leaves are closed, the embedment depth should be 2h or slightly larger than 2h. In one embodiment, the embedment depth 2h may be 1.5 cm to 4 cm, so as to ensure the shielding capacity of the opposite leaves against radiation beam when the opposite leaves are closed.

FIG. 4 (c) shows the leaf end design and the dimension of the end circular-arc surface according to another embodiment of the present disclosure, which corresponds to the leaf end design shown in FIG. 3. Similar to FIG. 4 (b), assuming the smaller of the distance between the position of the recess and the top surface and undersurface of the leaf is x, then the depth y of the recess may also be expressed as

y=d−2(R−\sqrt{R²−(l/2−x)²})

In a case where the two circular arc end surfaces of the opposite leaves are exactly completely embedded, the embedment depth d should be equal to 2h+c, wherein c is the length of the straight segment at the top edge, and this straight segment is connected to the arc segment. It can be calculated that the recess depth at the center of the leaf (i.e. the extension length of the protrusion at the center of the leaf) is 2h+c, and the recess depth at the edge of the leaf is c. In other words, the embedment depth between the opposite leaves at the center line of the leaves is twice larger than the contour height of the arc segment of the leaf end surface, which decreases to c towards the both edges of the leaf. In one embodiment, the length c of the above-mentioned straight segment may be 0.5-1.5 cm, and the embedment depth 2h+c may be 1.5 cm to 4 cm, so as to ensure that the opposite leaves maintain the shielding capacity against radiation beams within a larger adjustment range when they are closed.

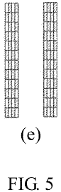

FIG. 5 shows the end design of the collimator leaf according to another embodiment of the present disclosure. As shown in FIGS. 5(a)-5(d), the collimator leaf of the present embodiment is similar to the leaf shown in FIG. 3, and comprise a main body and an end portion connected to the main body, with a similar end portion contour to the leaf contour shown in FIG. 3. The difference lies in the configuration structure of the protrusion and the recess at the leaf end portion. As shown in FIG. 5(e), the projection configuration of a plurality of recesses and a plurality of protrusions of the leaf end portion in a second plane perpendicular to the isocenter plane and the direction of leaf movement are disposed alternately in a lattice-like arrangement. In the figure, the blank area represent the protrusion, the shadow area represents the recess, and the protrusions and the recesses are configured alternately in a lattice mode in both the lateral and longitudinal direction. As shown in FIG. 5 (a) and FIG. 5 (e), there are 24 protrusions and 24 recesses at the end portion of each leaf, which are in interlaced distribution in both the lateral and longitudinal direction, so that the shielding capacity of the collimator leaves against radiation beams when the leaves are in closed state can be further improved.

Another embodiment of the present disclosure provides a radiotherapy apparatus, comprising a multi-leaf collimator of the type described above, and a controller, which may be configured to control the embedment degree of the end surfaces of the opposite first leaf and the second leaf at the closed position, so that the first leaf and the second leaf do not collide at the closed position. For example, the embedment depth between the opposite leaves may be controlled, e.g. by adjusting the size of the recess and the protrusion, and the range of leaf movement, in order to ensure that there will be no collision when the leaves are closed. By configuring the leaf end design as described above, the movement of the collimator leaf can be controlled within a large tolerance range without a direct contact between the first leaf and the second leaf, so as to ensure that the problems of the leakage radiation of the radiation and the dose gradual change area will not occur.

The basic principles of the present disclosure have been described above in connection with specific embodiments. However, it needs to be noted that merits, advantages, effects, and the like mentioned in the present disclosure are merely exemplary and not restrictive, and the merits, advantages, effects, and the like are not considered to be requisite in embodiments of the present disclosure. In addition, the specific details of the above application are only for the purpose of illustration and ease of understanding, and are not for a limiting purpose. The above details do not limit the present disclosure must be implemented with the above specific details.

In the present disclosure, words such as “including”, “comprising”, “having”, and the like are open-ended words, referring to “including but not limited to”, and may be used therewith interchangeably. The word “or” and “and” used herein refer to a word “and/or”, and may be used therewith interchangeably unless the context indicates otherwise clearly. The word “such as” used herein refers to a phrase “such as but not limited to”, and may be used therewith interchangeably.

The above is only the preferred arrangement mode of the present disclosure and is not intended to limit the present disclosure. Any modification, equivalent replacement, and improvement made within the spirit and principle of the present disclosure shall be included in the scope of the present disclosure.

What is claimed is:

1. A multi-leaf collimator for a radiotherapy apparatus, comprising: a plurality of pairs of leaves, leaves of each pair being disposed oppositely from each other and comprising a first leaf and a second leaf configured to move oppositely relative to each other, wherein, in a first plane perpendicular to an isocenter plane of the radiotherapy apparatus and parallel to a direction of leaf movement, the first leaf and the second leaf are capable of moving to a closed position, and end surfaces of the first leaf and the second leaf are embedded with each other at the closed position.

2. The multi-leaf collimator of claim 1, wherein the end surface is configured as a fold line contour composed of a plurality of recesses and a plurality of protrusions.

3. The multi-leaf collimator of claim 2, wherein the plurality of recesses and the plurality of protrusions of the end surface are alternately distributed.

4. The multi-leaf collimator of claim 2, wherein contour lines of the plurality of protrusions of the end surface are formed as an arc shape.

5. The multi-leaf collimator of claim 3, wherein projections of the plurality of recesses and the plurality of protrusions of the end surface in a second plane perpendicular to the isocenter plane and the direction of leaf movement are disposed alternately.

6. The multi-leaf collimator of claim 3, wherein projections of the plurality of recesses and the plurality of protrusions of the end surface in a second plane perpendicular to the isocenter plane and the direction of leaf movement are disposed alternately in a lattice-like arrangement.

7. The multi-leaf collimator of claim 1, wherein an end portion of the first leaf and/or the second leaf has a first thickness, and a main portion of the first leaf and/or the second leaf has a second thickness, the first thickness being greater than the second thickness.

8. The multi-leaf collimator of claim 7, wherein the first thickness of the first leaf and/or the second leaf decreases to the second thickness along a length direction from the end surface to the main portion of the first leaf and/or the second leaf.

9. The multi-leaf collimator of claim 1, wherein an embedment depth between the end surfaces of the first leaf and the second leaf at the closed position is calculated according to a distance from an embedment position to a center line of the pair of leaves.

10. The multi-leaf collimator of claim 9, wherein the embedment depth between the end surfaces of the first leaf and the second leaf at the center line of the pair of leaves is twice larger than a contour height of an arc segment of the end surface of the first leaf.

11. A radiotherapy apparatus, comprising:
a multi-leaf collimator comprising a plurality of pairs of leaves, leaves of each pair being disposed oppositely from each other and comprising a first leaf and a second leaf configured to move oppositely relative to each other, wherein, in a first plane perpendicular to an isocenter plane of the radiotherapy apparatus and parallel to a direction of leaf movement, the first leaf and the second leaf are capable of moving to a closed position, and end surfaces of the first leaf and the second leaf are embedded with each other at the closed position; and
a controller, configured to control an embedment degree of the end surfaces of the first leaf and the second leaf at the closed position, so that the first leaf and the second leaf do not collide at the closed position.
